(19) United States
(12) Patent Application Publication
(10) Pub. No.: US 2019/0388608 A1
(43) Pub. Date: Dec. 26, 2019

(54) MINIATURIZED MULTIPLE CHANNEL PRESSURE CONTROL SYSTEM

(71) Applicant: Percigenome, LLC, San Jose, CA (US)

(72) Inventors: Chen Li, Sunnyvale, CA (US); Yunfeng Ling, San Jose, CA (US); Cifeng Fang, Redmond, WA (US); Yaqi Wang, San Jose, CA (US)

(21) Appl. No.: 16/447,664

(22) Filed: Jun. 20, 2019

Related U.S. Application Data
(60) Provisional application No. 62/688,313, filed on Jun. 21, 2018.

(51) Int. Cl. A61M 5/142; F16K 99/00; F16K 11/10

(52) U.S. Cl. CPC A61M 5/142; A61M 2005/1401; F16K 11/105; F16K 99/0028

(57) ABSTRACT

Disclosed herein are systems comprising a pressure adjustment manifold, a pressure distribution manifold, and an electronic board, each of which are operatively connected. Also provided herein are methods of making and using the same.

This application claims priority to U.S. provisional application with the Ser. No. 62/688,313, filed Jun. 21, 2019.

### FIELD OF THE INVENTION

The field of the invention is pressure pump and medical device.

### BACKGROUND

The following description includes information that may be useful in understanding the present invention. It is not an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In microfluidic applications, syringe pumps are typically used for delivery of fluid. Regardless, the syringe pumps available for medical fluid delivery have several disadvantages. For example, while syringe pumps deliver fluid by flow rate control, some applications require precise pressure control. Moreover, syringe pump directly contacts sample fluid which could cause cross contamination from different samples. As such, multiple wash steps are required to reduce the contamination, which results in a longer total turnaround time. Even with such precautions, cross contamination is difficult to be totally avoided. Finally, integration of syringe pump involves lots of tubing, which makes integrated instrument cumbersome. In traditional pressure control system, pumps, regulators, and pressure sensors are used to control pressure. The system involves tubing for connection. In many traditional application cases, relatively large flows are involved. Usually one pump is used for one pressure supply; if multiple pressure control is desired, multiple sets of pumps, regulators, and sensors are used, making the system cumbersome.

Thus, there remains a need in the art for a compact pressure controller for microfluidic and medical instrument applications, which enables precise pressure control and avoids cross contamination efficiently.

### SUMMARY OF THE INVENTION

The disclosure herein provides in various embodiments, apparatuses, systems and methods comprising a compact portable device that uses one pump to generate multiple stable pressures without an outside pressure source.

Various embodiments of the instant disclosure include a system, comprising a pressure adjustment manifold, a pressure distribution manifold, and an electronic board, each of which are operatively connected to each other. The pressure adjustment manifold can comprise one or more of the following components: a pump, on-off valve, proportional valve, pressure sensor, orifice, and/or a fluid channel.

The pressure distribution manifold can comprise a 3-way valve and/or a fluidic channel. The fluid channel provides communication between one or more fluidic parts. It is contemplated that the electronic board controls the pumps, valves, and sensors in the system. Advantageously, the pump can provide pressure sources for two or more pressure output with different pressures. In some embodiments, one pressure outlet can be further split into two or more pressure output through pressure distribution manifold. The electronic board can communicate with pressure sensors and using PID control algorithm to control proportional valve for a desired pressure output.

In one embodiment, the system further comprises an outside pressure operatively connected to the system, which may be between 0 psi and 100 psi. Contemplated systems can have medical applications and/or microfluidic applications.

Embodiments of the present disclosure also include a method of using a pressure control system, comprising: providing a system having a pressure adjustment manifold, a pressure distribution manifold, and an electronic board, each of which are operatively connected; and using the pressure control system by (i) starting a pump in the pressure adjustment manifold, (ii) adjusting the valve in the pressure adjustment manifold to reach set pressures, and (iii) opening the valve in the pressure distribution manifold to obtain stable output pressures. In one embodiment, the pressure adjustment manifold has more than one valve. In one embodiment, the more than one valve outputs more than one pressure. In one embodiment, the method is for medical application. In one embodiment, the method is for microfluidic application.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates one embodiment of the compact pressure control system disclosed herein.

FIG. 2 illustrates a schematic of pressure adjustment manifold integrated with pump, valves, orifices, and pressure sensors.

FIG. 3 illustrates a detailed design for pressure adjustment manifold.

FIG. 4 illustrates pressure distribution manifold integrated with valves.

FIG. 5 illustrates one embodiment of detailed design of pressure distribution manifold.

FIG. 6 illustrates one example of two different channel pressure outputs.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

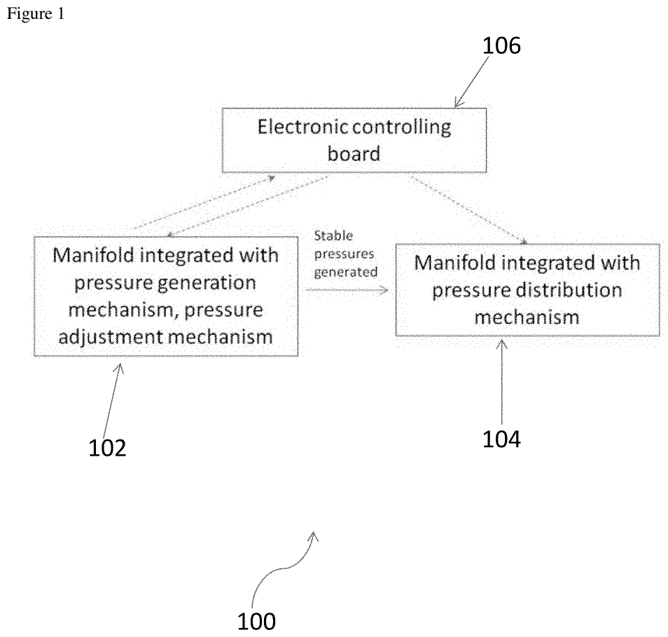

As illustrated in the schematic in FIG. 1, and in accordance to the various embodiments herein, the inventors have developed a compact pressure control system 100 that comprises three major blocks: manifold for pressure adjustment 102, manifold for pressure distribution 104, and electronic controlling board 106. In this compact pressure control system 100, the pressure adjustment mechanism 102, the pressure distribution mechanism 104, and the electronic controlling board 106, are each operatively connected to the others.

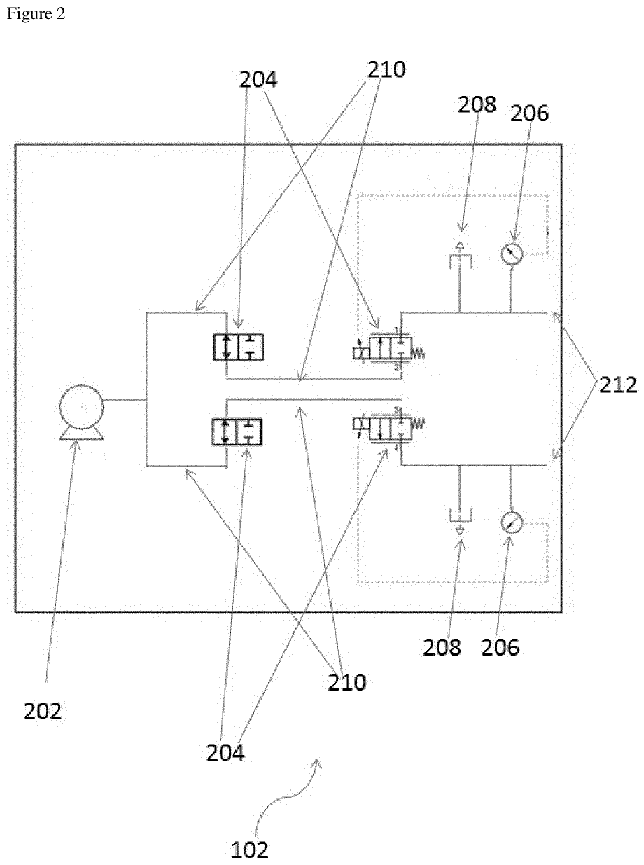

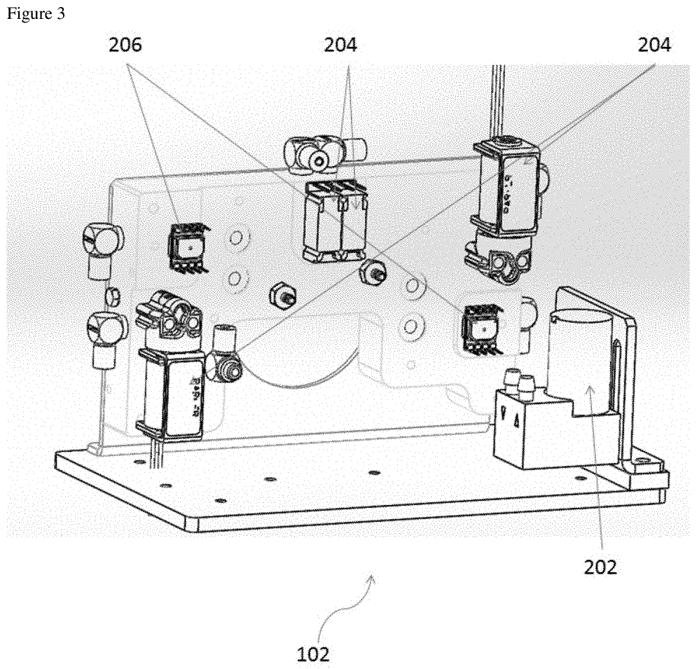

As illustrated in FIG. 2 and FIG. 3, the manifold for pressure adjustment 102 integrates pump 202, different kinds of valves 204, pressure sensors 206, orifices 208, and fluidic channels 210 as shown in the FIG. 2 (schematic) and FIG. 3 (a detailed design). In some instances, the valve may be a solenoid valve. In one embodiment, the overall manifold dimensions are 1.5 cm (height) by 10 cm (width) by 15 cm. The fluidic channels 210 inside the pressure adjustment manifold 102 provide connection between pump 202, valves 204, sensors 206, and orifices 208. The shape of fluidic channels can be circular, rectangular, trapezoidal, etc. The typical dimensions (diameter, height, or width) of fluidic channels are between 0.2 mm and 2 mm. The lengths of the fluidic channels 210 range between a few centimeters to tens of centimeters. The small volume design of fluidic channels 210 gives flexibility to integrate multiple channel pressure control using single low power consumption pump. The pump may be a diaphragm pump. As illustrated in FIGS. 2 and 3, one pump 202 is used to generate two different controlled pressure outputs 212. The number of different controlled pressure outputs 212 can be extended to three or more. In some instances a pump with higher power is used when there are multiple outputs.

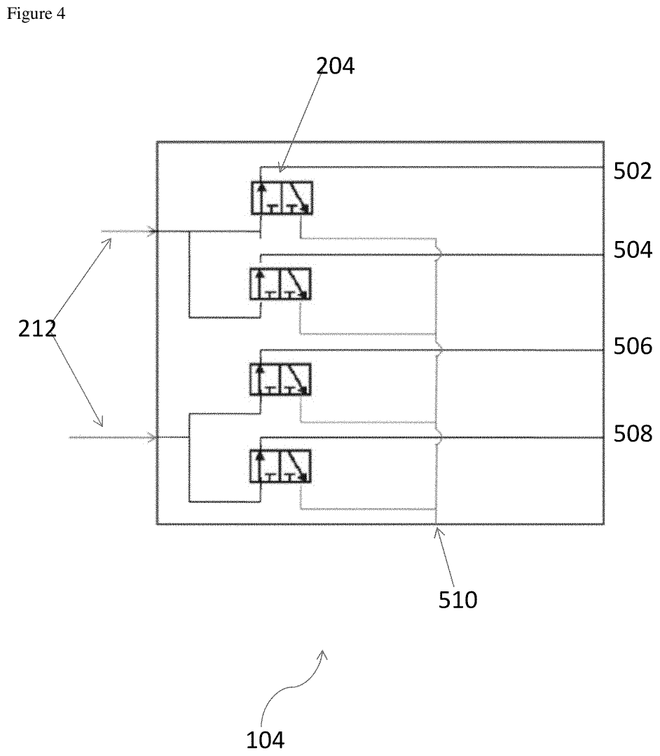

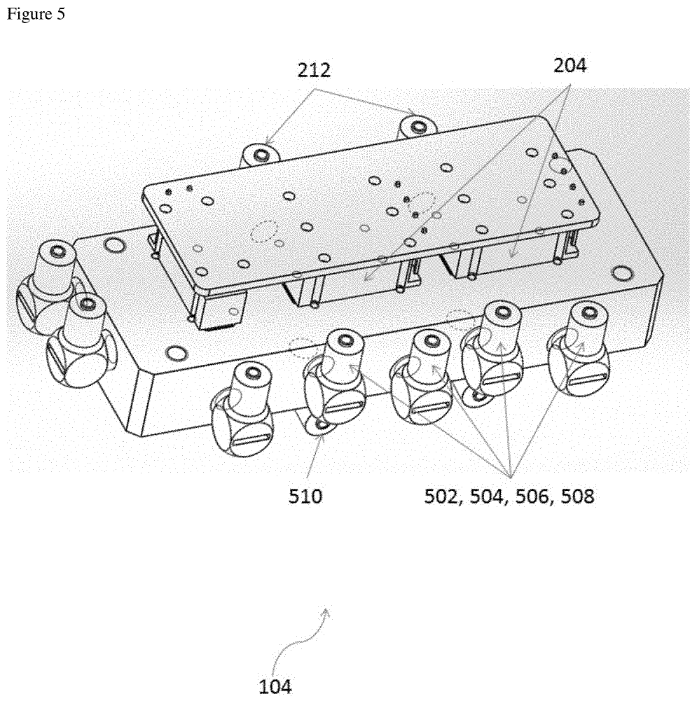

The electronic board 106 communicates with pressure sensors 206 and using PID control algorithm to control proportional valve 204 for a desired pressure output 212. In one embodiment, the pressure output 212 is between 0-20 psi. The pressure output 212 may also be vacuum which ranges from −10 psi to 0 psi. Multiple pumps 204 can be used for multiple pressure output (502, 504, 506, 508). In some instances, the pressure output ranges between 0-100 psi. In one embodiment, the pressure output accuracy is 0.1%. In one embodiment, the pressure control precision is 0.01 psi. The system can further comprise an outside pressure operatively connected to the system. For example, the outside pressure ranges between 0 psi and 100 psi. The pressure control system 100 disclosed herein has several uses, including for medical applications, and microfluidic applications. The pressure distribution manifold 104 used in the system is to further split one pressure output from manifold into multiple channels pressure output as shown in the FIG. 4 and FIG. 5. In this example, pressure output 1 from pressure adjustment manifold 102 further splits into two channels (pressure output A 502 and B 504), which are controlled by two 3-way valves 204. Output A 502 and B 504 either connect to air (1 atm) 510 or pressure input 1 212.

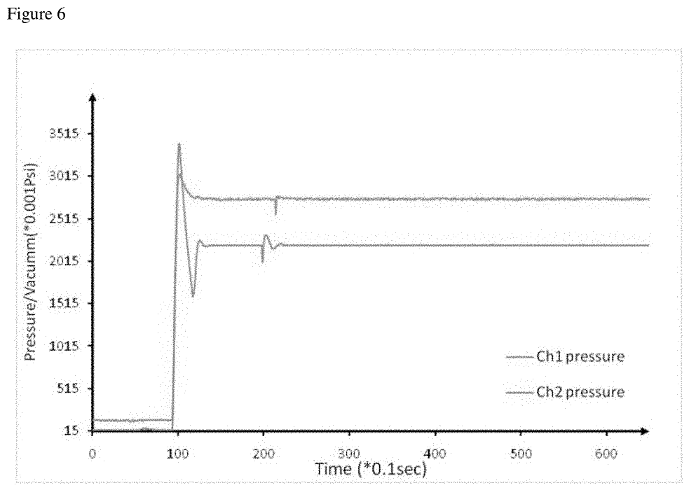

The electronic board 106 is used to communicate all the fluidic components and control them to get stable pressure output (502, 504, 506, 508) in a short time. Thus, the electronic board controls the pumps 202, valves 204 and sensors 206 in the system 100. FIG. 6 below shows two different channel pressures generated through this pressure control system 100. At around 10 s, the valves 204 on the pressure adjustment manifold 102 open and control system starts the pump and adjusts the proportional valves to reach set pressures (2.2 psi and 2.6 psi respectively in this case). Once the pressures are stabilized, the valves on pressure distribution manifold 104 open at around 20 s in this case, the pressures at output quickly reaches the set pressure quickly.

The pressure control system 100 disclosed herein may be used by starting a pump 202 in the pressure adjustment manifold 102, adjusting the valve 204 in the pressure adjustment manifold 102 to reach set pressures, and opening the valve 204 in the pressure distribution manifold 104 to obtain stable output pressures. In one embodiment, the pressure adjustment manifold has more than one valve 204. In one embodiment, the more than one valve outputs more than one pressure.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A system, comprising:
a pressure adjustment manifold, a pressure distribution manifold, and an electronic board, each of which are operatively connected to each other;
wherein the pressure adjustment manifold comprises one or more of the following components: a pump, on-off valve, proportional valve, pressure sensor, orifice, and/or a fluid channel;
wherein the pressure distribution manifold comprises a 3-way valve and/or a fluidic channel; and
wherein the electronic board controls the pumps, valves, and sensors in the system.

2. The system of claim 1, wherein the fluid channel provides communication between one or more fluidic parts.

3. The system of claim 1, wherein the pump is a diaphragm pump.

4. The system of claim 1, wherein the valves are solenoid valves.

5. The system of claim 1, wherein a pump provides pressure sources for two or more pressure output with different pressures.

6. The system of claim 1, wherein one pressure outlet can be further split into two or more pressure output through pressure distribution manifold.

7. The system of claim 1, wherein the fluidic channels can be shape of circle, rectangle, or trapezoid.

8. The system of claim 1, where the fluidic channel height or width ranges from 0.2 mm to 2 mm.

9. The system of claim 1, where the electronic board communicates with pressure sensors and using PID control algorithm to control proportional valve for a desired pressure output.

10. The system of claim 9, where the pressure output is between 0-20 psi.

11. The system of claim 9, where pressure output is vacuum which ranges from −10 psi to 0 psi.

12. The system of claim 9, where multiple pumps are used for multiple pressure output.

13. The system of claim 9, where the pressure output ranges between 0-100 psi.

14. The system of claim 9, where the pressure output accuracy is 0.1%.

15. The system of claim 9, wherein the pressure control precision is 0.01 psi.

16. The system of claim 1, further comprising an outside pressure operatively connected to the system.

17. The system of claim 16, wherein the outside pressure ranges between 0 psi and 100 psi.

18. A method of using a pressure control system, comprising:
providing a system having a pressure adjustment manifold, a pressure distribution manifold, and an electronic board, each of which are operatively connected; and
using the pressure control system by
(i) starting a pump in the pressure adjustment manifold,
(ii) adjusting the valve in the pressure adjustment manifold to reach set pressures, and
(iii) opening the valve in the pressure distribution manifold to obtain stable output pressures.

19. The method of claim 19, wherein the pressure adjustment manifold has more than one valve.

20. The method of claim 20, wherein the more than one valve outputs more than one pressure.
